(19) United States
(12) Patent Application Publication
(10) Pub. No.: US 2017/0113000 A1
(43) Pub. Date: Apr. 27, 2017

(54) CATHETER OR CANNULA ARRANGEMENT WITH FLOW SENSOR AND RELATED DEVICES, SYSTEMS, USES AND METHODS

(71) Applicants: Berlinger & Co. AG, Ganterschwil (CH); Star Syringe Limited, London (GB)

(72) Inventors: Corneliu Tobescu, Wilen (CH); Paul Mallins, Verbier (CH); William T. Denman, Winchester, MA (US)

(21) Appl. No.: 15/300,521

(22) PCT Filed: Apr. 1, 2015
(86) PCT No.: PCT/CH2015/000053

§ 371 Date: Sep. 29, 2016

(30) Foreign Application Priority Data
Apr. 1, 2014 (CH) 499/14

(51) Int. Cl. A61M 5/168; A61B 5/01; A61M 25/00; A61B 5/20; A61B 5/00

(52) U.S. Cl. CPC A61M 5/16886; A61B 5/208; A61B 5/6852; A61M 25/0017; A61B 5/01; A61M 2205/3334

(57) ABSTRACT

The catheter or cannula arrangement includes a catheter or cannula; a sensing unit including a sensing element for sensing a flow of a fluid; and an output unit operationally connectable to the sensing unit. The output unit is provided for receiving from the sensing unit signals related to the sensed flow of the fluid referred to as sensed signals, for obtaining, by processing the sensed signals, signals referred to as processed signals, and for outputting the processed signals, e.g., via a visual display. The output unit may include fixing structures attachment to tubing of the catheter or cannula. The sensing unit may be provided by a tube extension. The catheter or cannula may be a urinary catheter.

### BACKGROUND OF THE INVENTION

Field of the Invention

The invention relates to the field of medical devices and more specifically to catheter or cannula arrangements and constituents thereof as well as to related methods and uses. It relates in particular to methods and apparatuses, systems and uses according to the opening clauses of the claims. More particularly, it relates to flow measurement in catheter or cannula arrangements and/or to urinary catheters.

Description of Related Art

Catheter or cannula arrangements are very frequently used worldwide, for humans as well as for animals, in surgery and in other medical applications. Besides the catheter or cannula, they often comprise a needle inserted in and removable from the catheter or cannula.

Other catheter or cannula arrangements such as urinary catheters do not include a needle. Specific urinary catheters, more particularly those known as Foley catheters, have at least two separate channels, a first channel for guiding fluids, mainly urine, out of the body to which the catheter is applied, and a second channel for guiding another fluid into a balloon positioned inside the body, the enlarged balloon holding the inner end of the Foley catheter in place inside the body.

Furthermore, catheters are known that include a temperature sensing element for sensing the temperature inside the body. Such a catheter has an electrical connector, typically a cable with an electrical connector plug, by means of which the sensing element can be electrically connected to an external reading device which processes the signals output by the sensing element and displays the temperature sensed by the sensing element. Such catheters may find application, e.g., as urinary catheters for an early detection of an inflammation.

In addition, it is known to connect the above-mentioned first channel to an external device in which the drained fluid (mainly urine) is collected, and in which its flow rate is determined.

### SUMMARY OF THE INVENTION

The invention has originated from recognizing that it is possible and usually also desirable to miniaturize certain aspects related to sensing in conjunction with catheters and/or to make provisions making it possible to more simply obtain information on quantities sensed in conjunction with a catheter and/or to provide an improved mobility for a person or an animal with a catheter, in comparison with known devices.

More particularly, the invention originated from becoming aware of new possibilities for flow sensing in conjunction with urinary catheters. However, the new possibilities for flow sensing can also be applied in conjunction with other catheters or cannulas, e.g., with venous catheters such as peripheral venous catheters, or when blood or other fluids are guided out of a (human or animal) body in another way. The invention may find application in cardiovascular perfusion, in angiography, in dialysis, in arterial and in venous thrombectomy, in enteral feeding systems.

The fluid sensed usually is a liquid, at least predominantly.

In one aspect, the invention relates to sensing, in particular flow sensing, of fluids draining from a body or fluids coming out of a body—in contrast to fluids being injected into a body such as in case of intravenous infusions, e.g., of medicaments. For example, a flow of a fluid from a (urinary) bladder may be sensed, that fluid usually being composed prevailingly of urine, and a urinary catheter usually being used for that purpose. Or a flow of blood may be sensed, e.g., a flow of blood out of a blood vessel such as a vein, wherein usually a venous catheter will be used for that purpose, for example in case of a blood donation, or another body fluid is examined.

When a fluid is injected into a body, a person supervising the process is usually present. And the flow rate at which the fluid enters the body (and flows through the catheter or cannula) can usually be controlled and selected. However, in case of fluids leaving a body, this often is not the case. The flow rate of the fluid emitted is determined by the respective body, and, in particular in case of fluid emissions taking place and being monitored over long time periods (hours or even days), it is usually not practical to have a supervising person present all the time. Therefore, sensing a flow of a fluid leaving a body has very different requirements from flow sensing of injected fluids. However, the invention may, in one apect, relate to sensing, in particular flow sensing, of fluids being injected into a body such as in case of intravenous infusions, e.g., of medicaments.

Even though various aspects of the invention are described here in conjunction with urinary catheters (and related fluids), aspects of the invention may be applied also in case of other catheters or cannulas and/or also in conjunction with other fluids.

In principle, various flow sensing techniques may be applied. However, in order to facilitate flow sensing and/or for providing an improved mobility for a human or animal to whom the catheter or cannula is applied, it can be very advantageous to use a flow sensing technique which makes it possible to miniaturize a corresponding sensing element. For example, MEMS-based measuring, and in particular, thermal flow measurement is suitable. Thermal flow sensors are commercially available, e.g., from the company “Sensirion” (www.sensirion.com) and, in addition, they are operable in such a way that they are very insensitive to contamination by the fluid sensed.

Thermal flow sensing is usually based on a difference between a first and a second temperature measured in different locations along a flow path of the fluid. A thermal flow sensor may include at least one heating element and a temperature sensing unit for sensing temperature at at least two different locations, wherein with respect to a direction of flow, the heating element is located between these two locations. Accordingly, with respect to a direction of flow, before and after a heating zone, a temperature may be measured, and therefrom, information about a flow rate of the fluid can be obtained. A thermal flow sensor may include a volume which is surrounded by a cover and in which the fluid to be examined flows along a flow direction, and the temperature sensing and the heating is accomplished at the outside of the cover.

Other measurement principles such as, e.g., pressure-based or optical flow sensing may be suitable, too. However, below, the invention will be prevailingly described with reference to thermal flow sensing.

Another aspect of the invention relates to the power supply of the catheter or cannula arrangement. It can be desirable, for miniaturization and for increased mobility, to provide that the sensing can take place without a connection to an external power supply and more particularly to a connection to a mains supply or to a wall socket. In particular, the catheter or cannula arrangement may be powered (solely) by an energy storage unit such as by an energy storage unit including one or more batteries. E.g., the catheter or cannula arrangement may include no more than a single button cell (or perhaps two button cells), and this may be sufficient for powering the catheter or cannula arrangement.

In another aspect of the invention, the catheter or cannula arrangement includes a visual display. This makes possible to dispense with external visual displays. Accordingly, the results of sensing (“sensing results”) can be readily displayed, without having to bring close and connect to an external display. And it can make possible to study the sensing results while changing location (of the body to which the catheter or cannula is applied), e.g., when a patient goes for a walk, and, in addition, a person to whom the catheter or cannula is applied may, at virtually any time, check sensing results such as his urine flow rate or the amount of urine he emitted recently.

In another aspect of the invention, the catheter or cannula arrangement is structured and arranged for producing data representative of a graphic representation of sensing results (or, more precisely, of data representative of the sensing results). For example, a file containing such a graphic representation may be produced by the catheter or cannula arrangement. Such a file may have a commonly used format such as a format according to the PDF or GIF or JPEG standard. And, such a graphic representation (or, more precisely, the data representative of may be displayed by a visual display of the catheter or cannula arrangement (such as the above- or below-described visual display) and/or may be transmitted to an external device such as an external computing device, e.g., a tablet or laptop computer or a smart phone. It may be provided that the transmission is accomplished in a wirebound fashion and/or in a wireless fashion. For example, the catheter or cannula arrangement may include a data interface such as a USB interface. And/or, the catheter or cannula arrangement may include a wireless transmitter, e.g., a wireless transceiver, e.g., for transmission in the RF range (i.e. radio frequency range) of the data.

Such a graphics capability can simplify and speed up the analysis of sensing results.

In various cases, in particular in case of urinary catheters, the fluid emitted by the body to which the catheter or cannula arrangement is applied, is collected in a container. According to a current procedure, for monitoring the emitted fluid, the catheter or cannula is connected to a bulky external device. That external device has a housing accomodating the container as well as means for sensing the flow of the fluid and usually also a display for displaying sensing results. Because of the container alone, the external device is quite large and has, at least from time to time, a considerable weight. This usually impedes movment of the body to which the catheter or cannula arrangement is applied.

Furthermore, such an external device usually must be connected, directly or via a transformer or adapter, to a mains socket (cf. also the discussion of the energy supply above).

In another aspect of the invention, the catheter or cannula arrangement does not include a housing accomodating the container and, in addition, means for sensing the flow of the fluid and/or a display for displaying sensing results. Rather, there is no housing for the container. Or, if the container is in a housing, sensing takes place outside the housing and/or processing of sensed signals and/or display of sensing results takes place outside the housing.

This makes possible, e.g., that a patient wearing a permanent catheter and the container, is not obstructed in his movements, even when urine flow is measured.

In another aspect of the invention, the catheter or cannula arrangement includes an output unit for outputting sensing results. More specifically, the output unit is operationally connectable to the sensing unit, and it is structured and arranged
for receiving from the sensing unit signals related to the flow of the fluid referred to as sensed signals;
for obtaining, by processing the sensed signals, signals referred to as processed signals; and
for outputting the processed signals.

The processed signals may, e.g., include the above-mentioned sensing results.

The processed signals may, e.g., include the above-mentioned data representative of a graphic representation of sensing results.

In a wide interpretation of the term “processing”, said processing the sensed signals includes and may even be limited to processing of the sensed signals required for enabling said outputting the processed signals. In a narrower interpretation of the term “processing”, said processing the sensed signals exceeds a mere processing of the sensed signals required for enabling said outputting the processed signals.

The output unit may, e.g., include the before-mentioned visual display. However, it is alternatively possible to provide that the output unit is devoid of such a visual display and a corresponding display unit, respectively.

A first special aspect of the invention, which is closely related to the before-described aspect, relates to such an output unit or, more generally, to a device including an output unit. And in particular, the output unit may include one or more fixing structures for attaching the output unit to a catheter or cannula, in particular to tubing of a catheter or cannula—which may include an attaching to a tube extension of a catheter or cannula arrangement or, more specifically, to a tube extension of the catheter or cannula. By means of the fixing structures, such an output unit may be applied to existing catheters or cannulas, e.g., to standard urinary cathethers. And it may be reusable, e.g., such that one and the same person can continue using one and the same output unit while using, over time, different catheters or cannulas.

The output unit may furthermore include a connecting element for connecting to the sensing unit, wherein the sensing unit may be a sensing unit as described above or below. That connecting element may be, e.g., an electrical connector, and may, e.g., include an electric plug or a cable. Thus, a cable-bound electrical connection between the sensing unit and the output unit may be established via the connecting element. Alternatively or in addition, the connecting element may include a transmitter or transceiver for wireless transmission, e.g., in the RF range. Thus, a wireless connection between the sensing unit and the output unit can be established via the connecting element. The sensing unit, in this case, usually will have a receiver or transceiver for receiving corresponding transmitted signals.

It is also possible to provide that the output unit is integrated in the catheter or cannula. However, a detachable connection between output unit and catheter or cannula can make reuse of the output unit easier.

In another aspect of the invention, a sensing unit of the catheter or cannula arrangement including a sensing element for sensing a flow of a fluid is integrated in the catheter or cannula. More particularly, the sensing element is integrated in, e.g., fixedly attached to, tubing of the catheter or cannula.

In another aspect of the invention, which usually cannot be combined with the last-mentioned aspect, a sensing unit of the catheter or cannula arrangement including a sensing element for sensing a flow of a fluid is provided in a tube extension. The tube extension has two tubing ends or tubing connectors, one of them is connectable, in particular pluggable, to tubing of the catheter or cannula, the other to a container for collecting the fluid or to further tubing, wherein that further tubing usually is connectable, in particular pluggable, to the container for collecting the fluid.

For example: The tube extension may be plugged at one end to a tube end of the catheter or cannula, e.g., to an end of a tube of a urinary catheter through which urine is drained from the body to which the catheter is applied, and at the other end, the tube extension is plugged to tubing connected to the container for collecting the urine. The urine flow is sensed in the tube extension. And, e.g., the tube extension may be shorter than 20 cm, in particular shorter than 15 cm. It is also possible to provide, in the tube extension, a valve for blocking a flow of the fluid (e.g., urine) through the tube extension, wherein the valve may simultaneously be a valve for blocking a flow of the fluid (e.g., urine) through the sensing unit.

The valve may in particular be manually operable. The valve may be a rotary valve, but may alternatively be any other suitable type of manually operable valve.

A second special aspect of the invention, which is closely related to the before-described aspect, relates to such a tube extension. More particularly, it relates to a tube extension including a sensing element for sensing a flow of a fluid, and even more particularly, it relates to a tube extension including a sensing element for sensing a flow of a fluid and in addition including a valve for blocking a flow of the fluid (e.g., urine) through the tube extension, wherein the valve may simultaneously be a valve for blocking a flow of the fluid (e.g., urine) through the sensing unit.

The tube extension and in particular its sensing unit and also the valve may furthermore have properties as described elsewhere in the present patent application. For example, the sensing element may be a thermal flow sensor.

Furthermore, the sensing unit may include an connecting element for connecting to the output unit, wherein the output unit may be an output unit as described above or below. That connecting element may be, e.g., an electrical connector, and may, e.g., include an electric plug or a cable. Thus, a wire-bound electrical connection between the sensing unit and the output unit can be established via the connecting element. Alternatively, the connecting element may include a transmitter or transceiver for wireless transmission, e.g., in the RF range. Thus, a wireless connection between the sensing unit and the output unit can be established via the connecting element. The output unit, in this case, usually will have a receiver or transceiver for receiving corresponding transmitted signals.

A tube extension of the decscribed kind can be applied to existing catheters or cannulas, e.g., to standard urinary catheters.

Viewed from a particular point of view, which is rather closely related to the second special aspect described above, the invention relates to a flow sensing device including a sensing unit, the flow sensing device including a sensing element and a valve. The flow sensing device may furthermore have properties as described for the tube extension.

A third special aspect of the invention relates to a system (which may also be referred to as an arrangement) including an output unit and a sensing unit. And in particular, the output unit and the sensing unit may in this case be separate parts. The output unit and the sensing unit may, e.g., have a housing each, wherein these housings are distinct. The output unit and the sensing unit may have properties as described elsewhere in the present patent application.

This third special aspect may in particular relate to a system including an output unit according to the above-described first special aspect and a tube extension according to the above-described second special aspect.

In general, the output unit may in particular include one or more fixing structures for attaching the output unit to a catheter or cannula, in particular directly to tubing of the catheter or cannula.

The fixing structures may include clamping elements.

The fixing structures may include elements for partially encompassing tubing of the catheter or cannula.

By the fixing structures, a snap fit may be established between output unit and catheter or cannula.

By the fixing structures, a detachable connection may be established between output unit and catheter or cannula.

If the catheter or cannula has two or more channels, the fixing structures may be fixedly attached to tubing of at least two of the channels; more particularly, they may be attached to tubing of each of the at least two channels.

The fixing structures may be integrated in or fixedly attached to a housing of the output unit.

In particular, it may be provided that the output unit is—typically via the fixing structures—attached to tubing of the catheter or cannula in such a way that no further tubing is present between the output unit and the tubing of the catheter or cannula with the further tubing being arranged to be flown through by fluid which is to flow through the catheter or cannula, too.

A mechanical connection may be present between tubing of the catheter or cannula and the output unit (in particular its fixing structures), wherein this mechanical connection is absent tubing for guiding a fluid also guided by the catheter or cannula.

The sensing unit may be comprised in a tube extension.

Both, the output unit and the sensing unit are operationally connectable, e.g., via wire-bound connection, in particular wherein the wire-bound connection is pluggable at at least one end (at the output unit and/or at the sensing unit).

In a first particularly important aspect, the invention relates to a catheter or cannula arrangement, more particularly to a catheter or cannula arrangement including:
a catheter or cannula;
a sensing unit including a sensing element for sensing a flow of a fluid;

an output unit operationally connectable to the sensing unit, for receiving from the sensing unit signals related to the sensed flow of the fluid referred to as sensed signals, for obtaining, by processing the sensed signals, signals referred to as processed signals, and for outputting the processed signals.

In a second particularly important aspect, the invention relates to a catheter or cannula arrangement which is closely related to the above-described aspect in which said wide interpretation of the term “processing” may apply. More particularly, in this second important aspect, the invention relates to a catheter or cannula arrangement including:
a catheter or cannula;
a sensing unit including a sensing element for sensing a flow of a fluid;
an output unit operationally connectable to the sensing unit, for receiving from the sensing unit signals related to the sensed flow of the fluid referred to as sensed signals, and for outputting signals referred to as outputted signals, wherein the outputted signals are sensing results.

Therein, the outputted signals may in particular be sensed signals or signals referred to as processed signals, which are obtained by the output unit by processing the sensed signals.

Of course, also this second particularly important may be combined, as far as logically possible, with other aspects of the invention. For example:

In general and in particular also in this second particularly important aspect, the output unit may include one or more fixing structures for attaching the output unit to another part of the catheter or cannula arrangement, in particular to the catheter or cannula and/or to a tube extension including the sensing element. For example, the output unit may be attached by herein described fixing structures to related tubing.

And the catheter or cannula arrangement may, e.g., in addition, include an energy storage unit for supply of one or rather of both of the sensing unit and the output unit with electrical energy. The energy storage unit may be a battery. And the energy storage unit may be accomodated in a housing of the output unit.

Furthermore, in general and in particular also in this second particularly important aspect, the output unit may include a data storage unit, wherein at least one of
in the data storage unit, a sequence of flow data is stored, wherein each of the flow data of the sequence is representative of signals related to the sensed flow of the fluid at a different time;
the output unit includes a control unit structured and arranged for creating in the storage unit a sequence of flow data representative of a time development of signals related to the sensed flow of the fluid, in particular for creating the sequence of flow data by subsequently storing in the storage unit flow data representative of signals related to the sensed flow of the fluid at subsequent times.

This way, it is possible to provide that said sequence of data is outputted, e.g., to a display unit separate from the output unit, upon request, e.g., when a communication connection between the output unit and the display unit is established. Thus, at times when no such communication connection is present, the flow data can continually be gathered in the output unit and be stored and safely remain in the data storage unit.

Usually, the data sequence also includes also, for each of said flow data of the sequence, data representative of the time at which the respective flow data was obtained or at which the respective flow was sensed. This may be facilitated by providing that the catheter or cannula arrangement, more particularly the output unit, includes a clock providing real-time information.

Accordingly, the output unit may have the functionality of a data logger, logging sensing results such as the sensed signals or the processed signals or the outputted signals.

Thus, the output unit may include a control unit structured and arranged for transmitting said sequence of data to a display unit separate from the output unit. The display unit may then display a representation of the data sequence or of data derived from the data sequence. For example, a time-development of flow-related data can be displayed by the display unit.

When the catheter or cannula arrangement includes a display unit which is separate from and operationally connectable to the output unit, it may be provided that the display unit includes (and in particular is) at least one of:
a smart phone;
a handheld computer;
a tablet computer;
a laptop computer;
an intensive care monitoring device;
a wearable display device.

Furthermore, the catheter or cannula arrangement may comprise, in addition, a temperature sensing element for sensing a temperature inside a body of a person wearing the catheter or cannula, which is operationally connectable to the output unit. In this case, the output unit may in particular be structured and arranged for receiving from the temperature sensing element signals related to a temperature sensed by the temperature sensing element referred to as temperature signals, and for outputting signals referred to as temperature output signals. Therein, the temperature output signals are temperature signals or signals derived from temperature signals. As far as displaying (in particular visually displaying) the temperature output signals is concerned, the same may apply as for the flow signals (outputted signals, processed signals, cf. above and below), also as far as the outputting/transmitting of the signals (or data) is concerned. Of course, the temperature output signals may be combined with time-related signals in particular real-time data, for being able to find out which temperature had been sensed at which time.

Various more refined embodiments of the catheter or cannula arrangement are described throughout the present patent application.

In the catheter or cannula arrangement, it may in particular be provided that the output unit is arranged in a defined position relative to said catheter or cannula (referred to as initial position), in particular in a fixed position relative to said catheter or cannula. The output unit may, e.g., be clamped to tubing of the catheter or cannula; or it may be detachably attached to tubing of the catheter or cannula in another way.

In another aspect, the invention relates to a corresponding method, more particularly to a method for sensing a flow of a fluid flowing through a catheter or cannula applied to a body. Of course, that flow does not necessarily have to be sensed directly at the catheter or cannula (although it may be sensed there), but it may also be sensed elsewhere, in particular in or at tubing connected to the catheter or cannula, e.g., in or at a tube extension as described in the present patent application.

Specifically, it may be provided that in the method, a catheter or cannula arrangement is used which includes a catheter or cannula, a sensing unit including a sensing element, and an output unit operationally connected to the sensing unit. By means of the sensing element, the flow of the fluid is sensed, so-obtained signals (“sensed signals”) are transmitted to the output unit, the sensed signals are processed in the output unit, so as to obtain signals referred to as processed signals, and the processed signals are output by the output unit.

The sensing unit and the output unit may include separate housings, or may be comprised in one and the same housing.

It may furthermore in particular be provided that the sensed fluid is a fluid emitted, via the catheter or cannula, from the body to which the catheter or cannula is applied, e.g., a fluid drained from a urinary bladder.

In general, the invention includes methods with features of corresponding apparatuses (such as catheter or cannula arrangements, devices including an output unit, flow sensing devices, tube extensions and systems) according to the invention, and, vice versa, also apparatuses with features of corresponding methods according to the invention.

For example, the invention includes a method for sensing a flow of a fluid flowing through a catheter or cannula applied to a body, wherein the method includes using a catheter or cannula arrangement which includes a catheter or cannula, a sensing unit including a sensing element, and an output unit operationally connected to the sensing unit. The method furthermore includes:
A) sensing the flow of the fluid by means of the sensing element;
B) transmitting from the sensing unit to the output unit signals related to the sensed flow of the fluid referred to as sensed signals, wherein the transmitting is accomplished in a wireless or in a wirebound fashion;
C′) in the output unit, receiving the sensed signals;
D′) outputting from the output unit signals referred to as outputted signals, wherein the outputted signals are sensing results, in particular wherein the outputted signals are sensed signals or are signals referred to as processed signals, which are obtained by the output unit by processing the sensed signals.

In one embodiment, the method includes:
e) interconnecting a container for collecting the fluid and the catheter or cannula by means of a tube extension including the sensing unit.

In one embodiment, the method includes:
f) attaching the output unit to tubing of the catheter or cannula.

This may in particular be done by establising a snap fit between tubing of the catheter or cannula and one or more fixing structures of the output unit.

In one embodiment, the method includes:
g) establishing the operational interconnection between the output unit and the sensing unit.

This may in particular be done by establishing a wireless connection between the output unit and the sensing unit. On the other hand, this may be done by establishing a wire-bound connection between the output unit and the sensing unit. In the latter case, establishing the operational interconnection between the output unit and the sensing unit may include a plugging step.

From a specific viewpoint, the invention—and more specifically the method—relates to determining a quantity related to a flow of a fluid flowing through a catheter or cannula applied to a body. That quantity may be, e.g., a flow rate or an amount, e.g., a volume, of the fluid emitted from the body within a preset time interval, or a quantity related thereto.

Further aspects of the invention relate to uses of described apparatuses (such as catheter or cannula arrangements, devices including an output unit, flow sensing devices, tube extensions and systems) for sensing a flow of a fluid flowing through a catheter or cannula applied to a body or as a part of a catheter or cannula arrangement, the catheter or cannula arrangement including a catheter or cannula. More particularly, the uses can be uses for determining a quantity related to a flow of a fluid emitted from a bladder, such as a urinary bladder.

Therein, it may in particular be provided that the fluid is a fluid emitted, via the catheter or cannula, from the body to which the catheter or cannula is applied, and more particularly, that the fluid is predominantly urine, and the catheter or cannula is a urinary catheter, e.g., a Foley catheter.

Generally, the described aspects of the invention may be pair-wise combined or be combined in larger groups.

And the various apparatuses according to the invention—with exception of the catheter or cannula arrangement—may furthermore have features which are described for corresponding apparatuses as constituents of the catheter or cannula arrangement according to the invention.

It is an object of the invention to facilitate flow sensing in medical applications, in particular regarding fluids emitted from a body.

Another object of the invention is to provide improved catheter or cannula arrangements.

Another object of the invention is to provide an increased mobility for patients or animals with an applied catheter or cannula, in particular during flow measurements of drained fluid.

Another object of the invention is to provide wearable catheter or cannula arrangements with flow sensing capability.

Another object of the invention is to provide a way of making information concerning a sensed flow readily available, in particular without the need to use an external device therefor.

Another object of the invention is to provide an improved availability of flow-rate related data.

Another object of the invention is to provide or achieve an improved quality and/or information content of flow-rate related data.

Another object of the invention is to provide an improved documentation of flow-rate related data, in particular for patients and/or for health care persons.

Further objects emerge from the description and embodiments below.

Various aspects and embodiment of the invention have already been described above. However, further aspects, embodiments and advantages emerge from the patent claims, the description and the figures.

### BRIEF DESCRIPTION OF THE DRAWINGS

Below, the invention is described in more detail by means of examples and the included drawings. The figures show:

FIG. 1 a schematic illustration of a catheter or cannula arrangement with integrated sensing unit;

FIG. 2 a schematic illustration of a catheter or cannula arrangement with integrated sensing unit;

FIG. 3 a perspective view of a catheter or cannula arrangement with integrated sensing unit;

FIG. 4 a perspective view of the catheter or cannula arrangement of FIG. 3;

FIG. 5 a schematic illustration of a catheter or cannula arrangement including a tube extension including a sensing unit;

FIG. 6 a perspective view of a catheter or cannula arrangement including a tube extension including a sensing unit;

FIG. 7 a perspective view of a detail of the catheter or cannula arrangement of FIG. 6;

FIG. 8 a perspective view of a flow sensing device embodied as a tube extension including a sensing unit;

FIG. 9 a schematic illustration of a catheter or cannula arrangement including a tube extension including a sensing unit and an output unit, with attached container;

FIG. 10 a schematic illustration of a thermal flow sensing technique;

FIG. 11 a schematic illustration of an output unit and a system including the output unit;

FIG. 12 a schematical perspective illustration of a catheter or cannula arrangement including a display unit, which is separate from the output unit;

FIG. 13 a schematical perspective illustration of another catheter or cannula arrangement including a display unit which is separate from the output unit;

FIG. 14 a detail of the catheter or cannula arrangement of FIG. 13;

FIG. 15 a detailed view of the display unit of FIGS. 13, 14;

FIG. 16 a detail of the catheter or cannula arrangement of FIG. 12;

FIG. 17 a schematical perspective illustration of a detail of a catheter or cannula arrangement including an output unit, which is devoid of a visual display unit for displaying sensing results.

### DETAILED DESCRIPTION OF THE INVENTION

The described embodiments are meant as examples and shall not limit the invention.

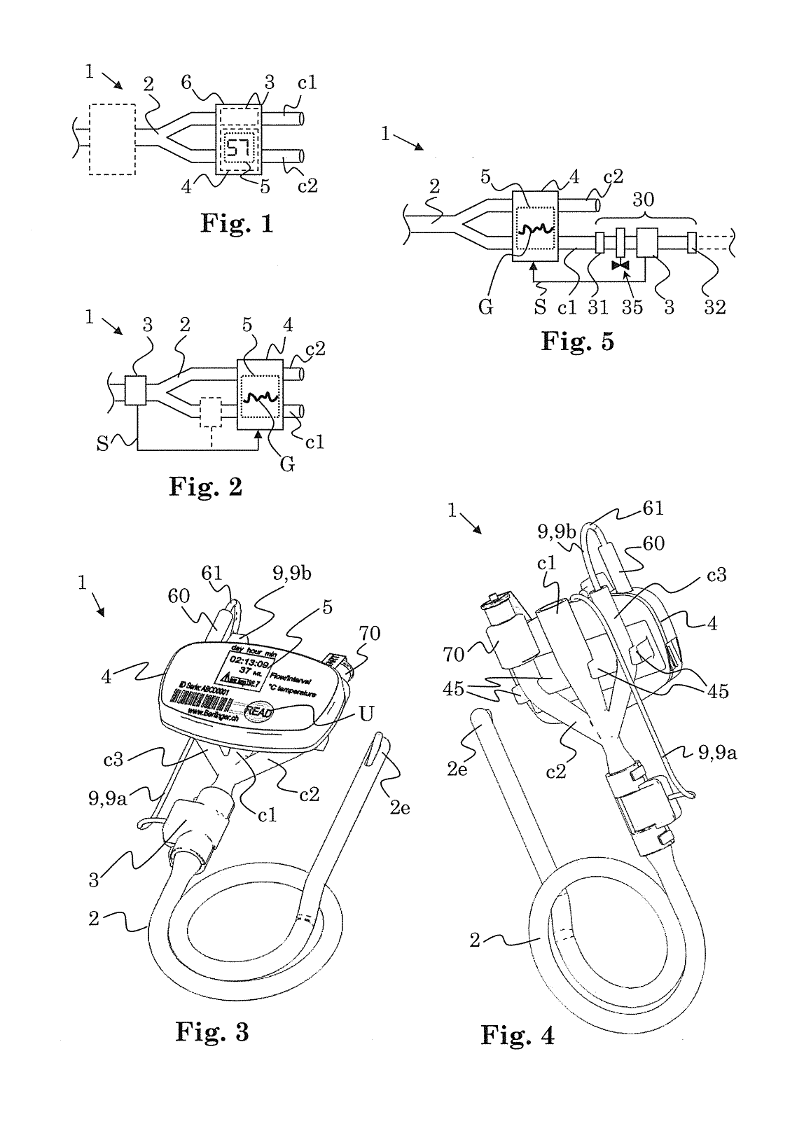

FIG. 1 is a schematic illustration of a catheter or cannula arrangement 1 with an integrated sensing unit 3. Catheter or cannula arrangement 1 includes a catheter or cannula 2 including tubing, sensing unit 3 and an output unit 4. In this embodiment, sensing unit 3 and output unit 4 share a common housing. The illustrated catheter or cannula 2 has two channels c1 and c2. However, it is also possible to provide a catheter or cannula having only one channel, such as a common venous catheter. And it is also possible to provide a catheter or cannula having three channels, such as a Foley catheter. The catheter or cannula 2 of FIG. 1 may be, e.g., a urinary catheter, and then, the fluid is predominantly urine.

Sensing unit 3 together with output unit 4 embodies a system 6 for flow sensing.

Sensing unit 3 includes a sensing element for sensing a flow of a fluid, such as a flow of a fluid flowing through channel c1 of catheter or cannula 2. Output unit 4 is operationally connected to sensing unit 3 and creates an output related to the sensed flow. For example, as illustrated in FIG. 1, output unit 4 optionally includes a display unit 5 such as a visual display 5, which may, e.g., as illustrated in FIG. 1, be an alphanumerical display, e.g., indicating a flow rate or an amount (such as a volume) of fluid which has flowed through sensing unit 3 within a selected time span such as 5 minutes.

Sensing unit 3 is attached to or rather integrated in catheter or cannula 2 and more particularly attached to or rather integrated in channel c1. However, as indicated by the dashed rectangle in FIG. 1, system 6 might also be attached to or rather integrated in a different position of catheter or cannula 2.

System 6 or the common housing—and thus also output unit 4—is attached to catheter or cannula 2, e.g., forming a snap fit between the housing and the tubing.

The small size of system 6 makes possible that the whole catheter or cannula arrangement 1 can easily be carried or worn by a patient to whom the catheter or cannula 2 is applied. And also in case of an application of the catheter or cannula to a body of an animal, mobility of the respective animal is not substantially restricted.

Furthermore, information about the flow is readily obtained using solely the catheter or cannula arrangement 1, no external device being needed.

FIG. 2 is a schematic illustration of another catheter or cannula arrangement 1 with an integrated sensing unit 3. This embodiment is in many regards very similar to the one of FIG. 1, thus, it is referred to there for details, but in the embodiment of FIG. 2, sensing unit 3 and output unit 4 have separate housings. Sensing unit 3 is integrated in catheter or cannula 2, and output unit 4 is arranged at channels c1, c2, e.g., clipped on the channels c1, c2.

This way, output unit 4 may readily be reused, even if catheter or cannula 2 and/or sensing unit 3 have to be discarded.

As illustrated in FIG. 2 by the dashed rectangle, sensing unit 3 may, alternatively, be present at and, more particularly, be integrated in channel c1.

The operational connection between sensing unit 3 and output unit 4 is illustrated by an arrow. Via this connection, signals (digital and/or analog) referred to as sensed signals S are transmitted.

Visual display 5 may, e.g., display a graphic representation G of data, such as, e.g., a time development of the flow of the sensed fluid.

In output unit 4, sensed signals S are processed, so as to obtain data referred to as processed data, which are, e.g., data suitable for displaying in visual display 5. However, the processing may also include integrating sensed signals and performing various calculations and the like.

FIG. 3 is a perspective view of a catheter or cannula arrangement 1 with integrated sensing unit 3. The catheter or cannula 2 is different from the one illustrated in FIGS. 1 and 2, in that it is a three channel catheter or cannula 2. More particularly, in FIG. 3, the catheter or cannula 2 is a Foley catheter with three channels c1, c2, c3, and having an inner end 2e to be present inside a body and at which a balloon can be inflated. Otherwise, the embodiment of FIG. 3 corresponds largely to the one of FIG. 2, please refer to there for further details.

The operational connection between sensing unit 3 and output unit 4 is embodied in FIG. 3 as an electrical connection including a connector 9 including a cable 9a and a plug 9b, wherein plug 9b is plugged into output unit 4.

Output unit 4 optionally has a user interface U, e.g., including a button. User interface U is provided, e.g., for selecting which data shall be outputted by output unit 4, or for selecting the length of a time interval during which sensed fluid amounts shall be integrated.

Channel c2 is provided for guiding a fluid (typically water) into the balloon at end 2e of catheter 2. Channel c2 is provided with a valve unit 70 for opening and closing channel c2.

Channel c3 is provided for temperature sensing. At or near end 2e, a temperature sensing element is present (not illustrated), and corresponding signals are fed to output unit 4. For example, a cable 61 running through channel c3 is provided with a plug 60 plugged into output unit 4.

User interface U might furthermore make possible to select whether temperature-related data or flow-related data shall be displayed. The displayed information may, of course be related to real time information, as herein described.

FIG. 4 is a perspective view of the catheter or cannula arrangement 1 of FIG. 3. This view shows that output unit 4 includes fixing structures 45 by means of which output unit 4 is attached to catheter or cannula 2, more particularly to tubing therof.

In general, it can, for catheters or cannulas with more than one channel, be advantageous to fix output unit 4 to a catheter or cannula 2 in a region where at least two of the channels have separate tubing, e.g., as illustrated in FIGS. 3 and 4. This may provide an increased stability of the mechanical connection between output unit 4 and catheter or cannula 2.

Fixing structures 45 illustrated in FIG. 4 include clamping elements partially encompassing the tubing of each of channels c1, c2 and c3. A detachable connection and more particularly, a snap fit is established between output unit 4 and the tubing of catheter or cannula 2.

FIG. 5 is a schematic illustration of a catheter or cannula arrangement 1 including a tube extension 30 including a sensing unit 3. The embodiment of FIG. 5 is similar to the one of FIG. 2 in that sensing unit 3 and output unit 4 are separately arranged parts and in that output unit 4 is attached to tubing of the catheter or cannula 1. But in the embodiment of FIG. 5, sensing unit 3 is not integrated in catheter or cannula 1.

Sensing unit 3 is included in a tube extension 30 or can be considered to be embodied as a tube extension 30. And accordingly, tube extension 30 can also be considered a flow sensing device.

Tube extension 30 is connected to, more particularly plugged to, catheter or cannula 2, more particularly to the outer end of channel c1. For this purpose, tube extension 30 has a first tubing connector 31. At its opposite end, tube extension 30 has a second tubing connector 32 to which further tubing may be connected. It would also be possible to provide that tube extension 30 has, at one or both ends, tubing ends, and to provide a (separate) tubing connector for establising tubing connections.

It is possible to provide that output unit 4 is attached to the tube extension 30, in particular to tubing thereof. This can, in general (i.e. in other embodiments, too), be an alternative to having output unit 4 attached to catheter or cannula 2. Fixing structures can be embodied as described above or below.

FIG. 5 also shows a valve 35. A valve is, in general, an optional feature of a tube extension including a sensing unit, which may in several applications be advantageous, e.g., because it may make possible to miniaturize the catheter or cannula arrangement 1 and/or to increase the functionality of the tube extension. By means of valve 35, which typically is manually operable, a flow of fluid through tube extension 30 and through sensing unit 3 can be blocked (or allowed). This can be valuable, e.g., in case a container for collecting drained urine has to be exchanged or emptied.

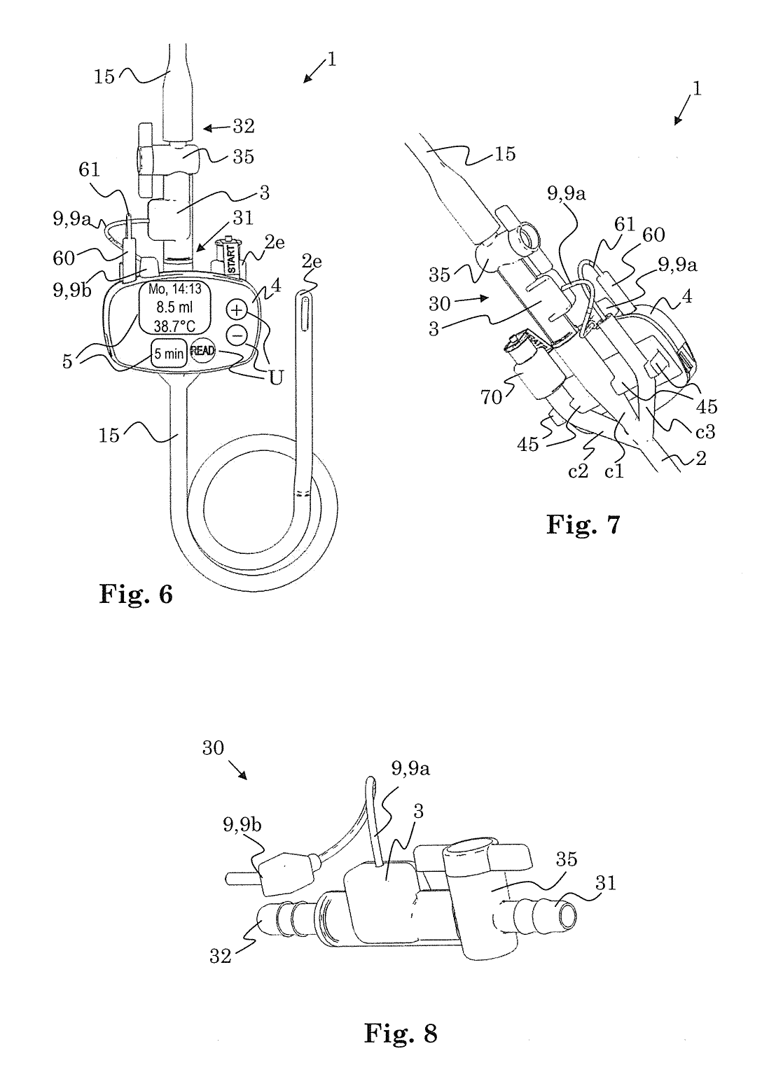

FIG. 6 is a perspective view of a catheter or cannula arrangement 1 including a tube extension 30 having a sensing unit 3, and FIG. 7 is a perspective view of a detail of the catheter or cannula arrangement 1 of FIG. 6.

The catheter or cannula arrangement 1 of FIGS. 6 and 7 can be considered a more concrete embodiment of the one of FIG. 5, with the exception that—like in FIGS. 3 and 4—three instead of two catheter channels are provided. The valve 35 is a rotary valve, for ease of use, although any other suitable type of valve could be used.

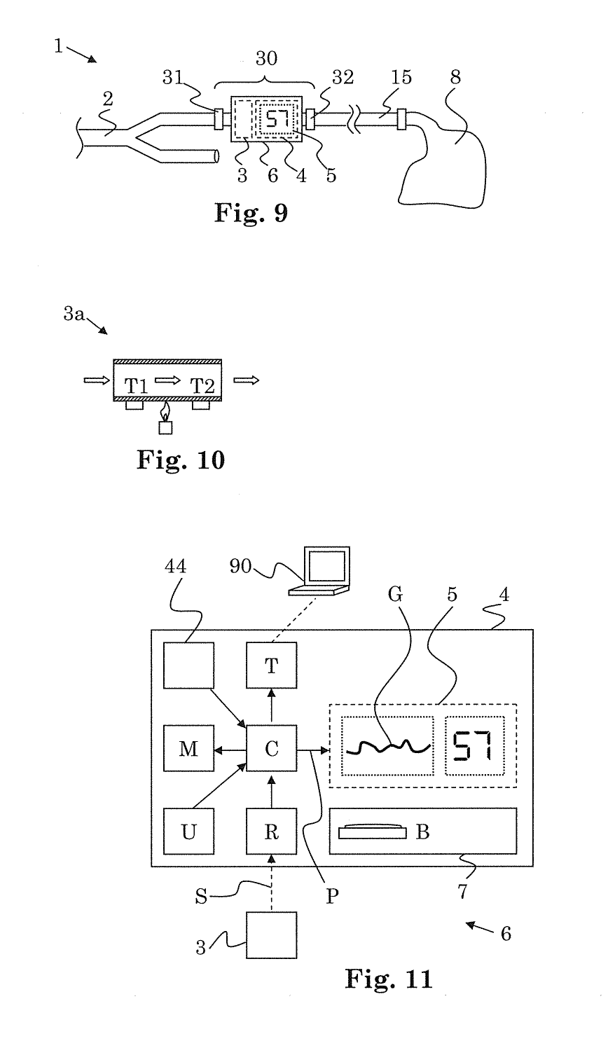

At its tube connector 32, tube extension 30 is connected to further tubing 15. At the end of tubing 15, a container like the before-mentioned one or the one of FIG. 9 (cf. below) may be connected.

The output unit 4 illustrated in FIGS. 6 and 7 is similar to the one of FIGS. 3 and 4 and is connected to the tubing of catheter or cannula 2 in a similar way, but the visual display 5 and the user interface U are embodied differently. However, also in FIGS. 6 and 7, visual display 5 and user interface U are merely optional.

FIG. 8 is a perspective view of a flow sensing device 30 embodied as a tube extension including a sensing unit 3. It may be identical to the one illustrated in FIGS. 6 and 7.

Such a tube extension or flow sensing device 30 can be very compact and may be used with known catheters or cannulas.

In FIG. 8, the tube extension or flow sensing device 30 has two ends with a tubing connector 31,32 each, but it may be provided that one or both ends are embodied as tubing ends instead.

FIG. 9 is a schematic illustration of a catheter or cannula arrangement 1 including a tube extension 30 including a sensing unit 3 and an output unit 4, with a container 8 connected, for collecting drained fluid. Such a container may, of course, be connected in case of any one of the other embodiments as well. Further tubing 15 is provided for connecting container 8, which may, e.g., be a bag, to tube extension 30.

The catheter or cannula arrangement 1 of FIG. 9 is similar to the one of FIGS. 5, 6 and 7 in that it includes a tube extension 30 including or, even, constituting a sensing unit 3. And the catheter or cannula arrangement 1 of FIG. 9 is similar to the one of FIG. 1 in that it includes a system 6 for flow sensing containing sensing unit 3 and output unit 4 in one common housing.

Tube extension 30 may be considered a flow sensing device. It may otherwise be embodied as described above, cf., e.g., FIGS. 5 to 8, and it may, too, include a valve such as valve 35 in FIGS. 5 to 8 (not illustrated in FIG. 9).

Flow sensing may thus be accomplished based on a known catheter or cannula 2 merely by providing the tube extension 30, with no further additions of modifications to the catheter or cannula 2 necessary.

There are various possibilities for the sensing technique for flow sensing, some of them have been mentioned above. Of particular interest is, however the thermal flow sensing technique. The degree of miniaturization achievable in this technique is very high, making possible to manufacture very small sensors.

FIG. 10 is a schematic illustration of a thermal flow sensing technique. In such a sensing technique, the fluid to be sensed flows through a volume V, as indicated by the open arrows, which also indicate the direction of flow. A heating device (symbolized in FIG. 10 by a candle) and at least two temperature sensing elements T1, T2 are provided, at least one (T1), with regard to the direction of flow, before, and at least one (T2), with regard to the direction of flow, after the position of the heating device. From differences in temperature sensed by the temperature sensing elements, estimations regarding the flow rate of the fluid can be made. With suitable gauging provided, precise absolute flow rate values can be obtained, at least if the fluid is of a sufficiently stable composition.

The temperature sensing elements T1, T2 may be, and usually are, located outside the volume V, such that contamination can effectively be prevented and long-term stability be achieved. A cover such as tubing may be present between volume V (and thus the fluid to be sensed) and the temperature sensing elements T1, T2.

Temperature sensing elements T1, T2 and the heating device may be embodied in a single semiconductor chip.

FIG. 11 is a schematic illustration of an output unit 4 and a system 6 including the output unit 4. Such an output unit 4 may be provided in any of the described embodiments, independent, e.g., of sensing unit 3 and output unit 4 being present in a common housing or in separate housings. FIG. 11 emphasizes functionalities realized in output unit 4.

Output unit 4 includes an interface R for receiving sensed signals S from a sensing unit 3. In case of a wireless connection between sensing unit 3 and output unit 4, this may be a wireless receiver or transceiver, in case of wire-bound connection, interface R may be realized in a simpler fashion.

Interface R feeds signals S or signals derived therefrom to a control unit C, which may also be considered a processing unit. Control unit C may store those signals and/or signals derived therefrom (by processing in control unit C) in a data storage unit M of output unit 4. In particular, control unit C may, e.g., integrate signals, e.g., in order to obtain time-averaged flow-related values, and/or produce signals displayable in a display unit such as visual display 5 of output unit. For example, numerical output and/or graphical output may be computed and displayed. However, visual display 5 is generally optional. In one embodiment, output unit 4 includes no such display unit. In this case, a display unit 5, in particular a visual display, may be, e.g., provided in another component of the catheter or cannula arrangement or in an external device 90 (cf. below).

Data storage unit M makes possible to store in output unit 4 data representative of sensed signals, of sensing results, of processed data or in general data related to the sensed flow, in particular such that such data taken at some time can be outputted, e.g., transmitted, by output unit 4 at a later time. For example, data can be gathered in output unit 4 and transmitted elsewhere, e.g., to an external display unit, upon request only. This can make the flow measurement safer and can contribute to an increased mobility of a person wearing the catheter or cannula and the output unit 4.

Output unit 4 also includes a clock 44, in particular wherein clock 44 provides real-time information, such as at least the hour of the day, usually also, in addition, the day of the week and/or the full date. This way, sensed flow-related data can be linked to the time of sensing. Thus, it is possible to obtain (and store) information such as “today, between 11 a.m. and 12 a.m., 89 ml urine were collected” or “yesterday, between 11 a.m. and 12 a.m., 48 ml urine were collected”.

Output unit 4 has the functionality of a data logger. Flow-related data are logged in output unit 4, possibly in addition to temperature-related data. Of course, one or (rather) both of the sensed flow-related data and sensed temperature data can be linked to the time of the respective sensing.

Output unit 4 includes an interface T for outputting processed flow-related data, as obtained by processing in control unit C. In case of a wireless connection between output unit 4 and an external device 90 such as a computer, e.g., a tablet computer, this may be wireless transmitter or transceiver; in case of wire-bound connection, interface T may be realized in a simpler fashion, wherein it is in particular possible to provide a digital interface, e.g., according to the widely used USB standard.

Output unit 4 includes its own power supply, usually in form of an energy storage unit 7 such as a battery, e.g., a button cell. Thus, output unit 4 and therefore typically also the corresponding system 6 and the catheter or cannula arrangement 1 does not require a connection to a mains socket. This provides mobility to the person or animal wearing the catheter or cannula. And output unit 4 may have very small outer dimensions.

Optionally, a user interface U is provided, e.g., by one or more buttons, e.g., for selecting data to be displayed and/or outputted by output unit 4, or for setting a length of a time interval during which integration of sensed signals shall be accomplished.

As has been mentioned already, it can, in general, also be envisaged to dispense with a visual display 5. Then, it is not possible anymore to be readily visually informed about the sensed flow by output unit 4, however, corresponding information may be displayed using a unit connectable (via interface T) to output unit 4, e.g., to a smart phone or to a tablet computer or to an intensive care monitoring device wirelessly connected to output unit 4. More particularly, it is possible to provide that in output unit 4, more particularly by means of control unit C, files are generated which contain graphics data, such as data representative of a graphic visualization of flow-related data.

Further details concerning processing have been described above and therefore need not be repeated here.

However, a catheter or cannula arrangement may include a display unit 5 that is separate from output unit 4. Then, a display unit 4 is not integrated in output unit 4. In particular, output unit 4 and display unit 5 may each have their own housing.

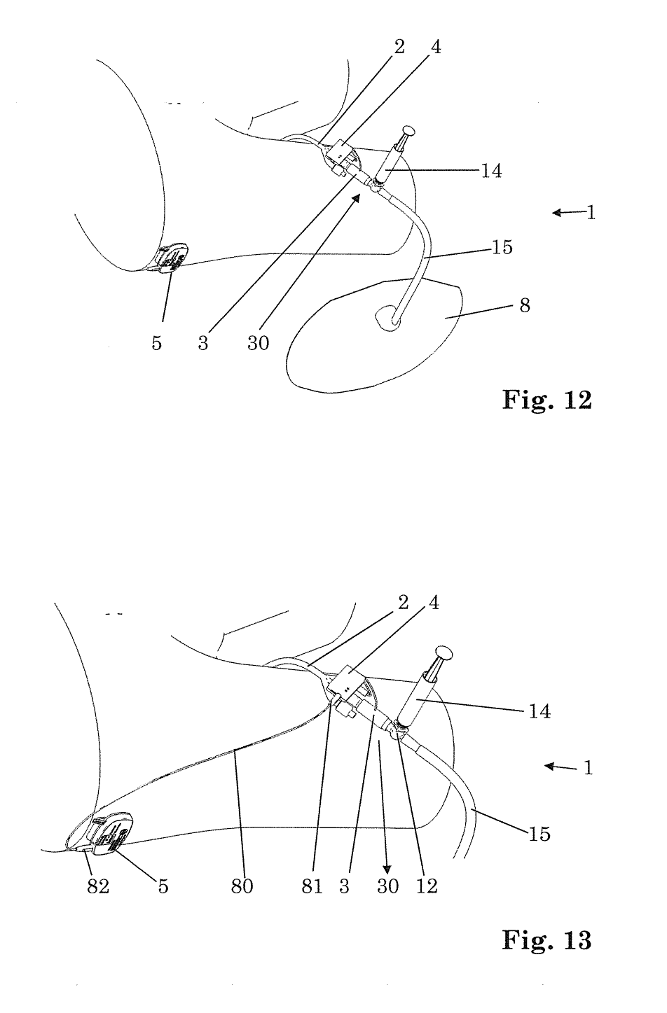

FIG. 12 is a schematical perspective illustration of a catheter or cannula arrangement 1 including a display unit 5 which is separate from the output unit 4. A torso is symbolized in FIG. 12 to illustrate how and where the components of the catheter or cannula arrangement 1 may be worn. Output unit 4 is attached to tubing, more specifically to tubing 2 of the catheter or cannula, but it could also be attached to a tube extension or flow sensing device 30 including sensing unit 3.

Urine is guided by further tubing 15 to bag 8.

In FIG. 12, furthermore a syringe 14 is illustrated which is used for taking urine samples from tube extension or flow sensing device 30 having a port suitable therefor.

In the embodiment of FIG. 12, a transmission of outputted data outputted by output unit 4 to display unit 5 is accomplished in a wireless fashion.

FIG. 13 shows, in a similar fashion as FIG. 12, a schematical perspective illustration of another catheter or cannula arrangement 1 including a display unit 5 which is separate from the output unit 4. The arrangement is similar to the one of FIG. 12, but the data transmission between output unit 4 and display unit 5 is accomplished in a wirebound fashion, e.g., via illustrated cable 80 and connectors or plugs 81, 82.

For both cases, wireless (FIG. 12) and wirebound (FIG. 13) data transmission, the transmitted data are usually flow-related data, in particular sensing results. Temperature-related data may be transmitted, too. Sensed signals from sensing unit 3 received in the output unit 4 may be merely processed for enabling the transmission or may be processed in excess thereto, e.g., for obtaining gauged flow rate values from the sensed signals, or even for producing data representative of a graphic representation.

Output unit 4 may furthermore receive temperature data from a temperature sensor, cf. the description of FIG. 3, which is not illustrated in FIGS. 12, 13. Accordingly, data outputted by output unit 4 may include temperature-related data, more specifically data related to or, rather, indicative of, a temperature inside the body of a person wearing the catheter or cannula arrangement.

Display unit 5 may include a visual display such as an LCD panel. By means of the visual display, flow-related data as well as temperature-related data may be displayed, e.g., in relation to the respective time of sensing.

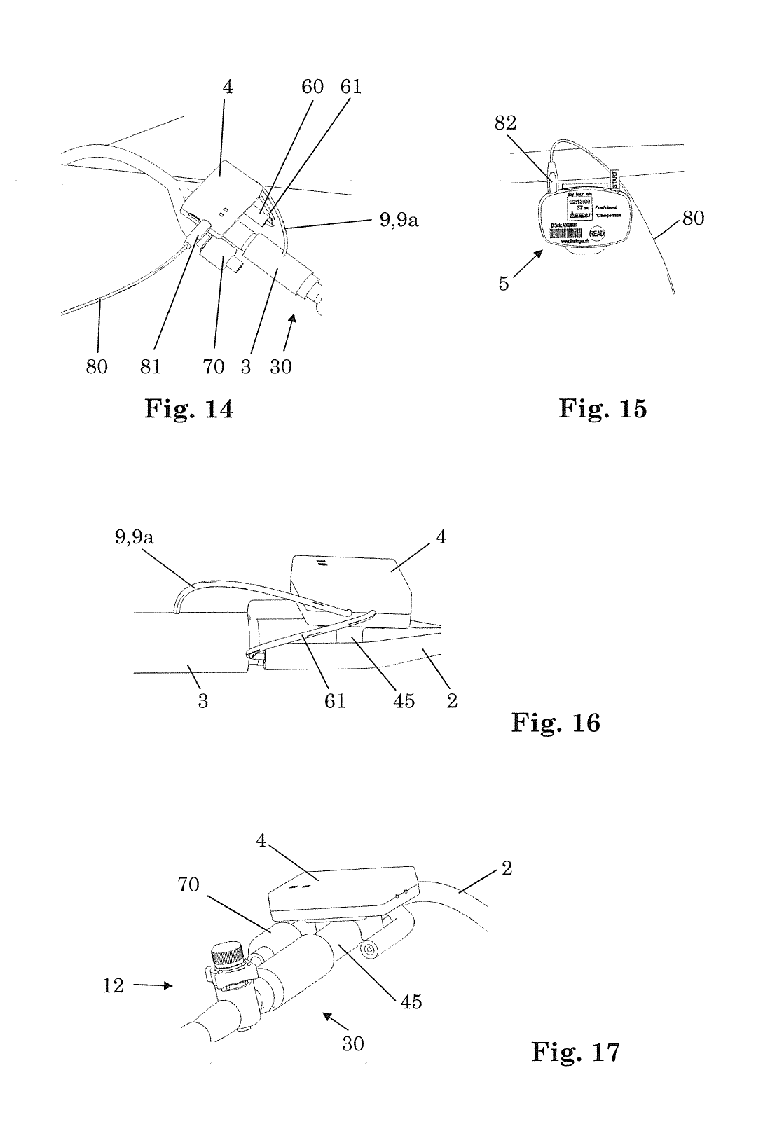

FIG. 14 shows a detail of the catheter or cannula arrangement of FIG. 13. Wired connections between output unit 4 and sensing unit 3 (cf. at 9, 9a) and between output unit 4 and the temperature sensor (cf. at 60, 61) are illustrated. Generally, a wireless communication connection between output unit 4 and sensing unit 3 and/or between output unit 4 and the temperature sensor is possible, too.

FIG. 15 is a detailed view of the display unit 5 of FIGS. 13, 14. The display unit includes a user interface having a visual display, wherein the user interface may, in addition, be structured and arranged for receiving user input. The user interface may have the same properties as described above, cf., e.g., FIG. 3, for the user interface U provided in an output unit 4. In that case, output unit 4 may be devoid of any user interface or, rather, devoid of any user interface for receiving user input.

FIG. 16 shows a detail of the catheter or cannula arrangement 1 of FIG. 12 in which fixing structures 45 are illustrated.

FIG. 17 is a schematical perspective illustration of a detail of a catheter or cannula arrangement including an output unit 5, which is devoid of a visual display unit for displaying sensing results. Cables are not illustrated in FIG. 17. Tube extension or flow sensing device 30 includes a port 12 as already mentioned above (cf. FIG. 12) which permits to access urine inside tube extension or flow sensing device 30, in particular by means of a syringe, e.g., by screwing the syringe onto port 12.

In the embodiments of FIGS. 12 to 17, output unit 4 may have, as illustrated, status indicator lights such as two LEDs, e.g., for indicating that output unit is switched on and powered and/or for indicating that signals are received in output unit 4 and/or for indicating that signals are outputted by output unit 4.

Aspects of the embodiments have been described in terms of functional units, in particular in case of FIG. 11. As is readily understood, these functional units may be realized in virtually any number of hardware and/or software components adapted to performing the specified functions. For example, interfaces R and T may be realized in a single unit and in a single semiconductor chip. Or control unit C and data storage unit M may be realized in a single semiconductor chip, possibly together with clock 44.

The invention has the potential to strongly influence the way flow sensing is accomplished in conjunction with catheters or cannulas and more particularly how sensing of a quantity related to a flow of a fluid emitted from a body is accomplished. It can become possible to readily monitor a flow of such a fluid over a long time. And the respective flow sensing may become simpler and more cost-effective.

Catheter or cannula arrangements described in the present patent application may be worn by the respective person or animal providing increased comfort and mobility in comparison with current arrangements.

1-72. (canceled)

73. A catheter or cannula arrangement, comprising
a catheter or cannula;
a sensing unit comprising a sensing element for sensing a flow of a fluid;
an output unit operationally connectable to the sensing unit, for receiving from the sensing unit signals related to the sensed flow of the fluid referred to as sensed signals, and for outputting signals referred to as outputted signals, wherein the outputted signals are sensing results.

74. The catheter or cannula arrangement according to claim 73, wherein the outputted signals are sensed signals.

75. The catheter or cannula arrangement according to claim 73, wherein the outputted signals are signals referred to as processed signals which are obtained by the output unit by processing the sensed signals.

76. The catheter or cannula arrangement according to claim 73, wherein the output unit comprises one or more fixing structures for attaching the output unit to another part of the catheter or cannula arrangement.

77. The catheter or cannula arrangement according to claim 73, wherein the output unit comprises one or more fixing structures for attaching the output unit to tubing of the catheter or cannula.

78. The catheter or cannula arrangement according to claim 73, comprising an energy storage unit for supply of at least one of the sensing unit and the output unit with electrical energ, and wherein the energy storage unit is comprised in the output unit.

79. The catheter or cannula arrangement according to claim 73, wherein the output unit comprises a data storage unit, and wherein at least one of
in the data storage unit, a sequence of data is stored, wherein each of the data of the sequence is representative of signals related to the sensed flow of the fluid at a different time;
the output unit comprises a control unit structured and arranged for creating in the storage unit a sequence of data representative of a time development of signals related to the sensed flow of the fluid and for creating the sequence of data by subsequently storing in the storage unit data representative of signals related to the sensed flow of the fluid at subsequent times.

80. The catheter or cannula arrangement according to claim 73, wherein the output unit and the sensing unit have separate housings.

81. The catheter or cannula arrangement according to claim 73, wherein the output unit is
devoid of any visual display capable of graphically or alphanumerically displaying information; and
devoid of any visual display structured and arranged for graphically or alphanumerically displaying a representation of sensed signals or of data derived from sensed signals.

82. The catheter or cannula arrangement according to claim 73, comprising a display unit which is separate from and operationally connectable to the output unit, wherein the output unit is structured and arranged for transmitting the outputted data to the display unit, and wherein the display unit is structured and arranged for receiving from the output unit the outputted signals and for visually displaying a representation of sensing results.

83. The catheter or cannula arrangement according to claim 82, wherein the display unit comprises at least one of
a smart phone;
a handheld computer;
a tablet computer;
a laptop computer;
an intensive care monitoring device;
a wearable display device.

84. The catheter or cannula arrangement according to claim 82, comprising, in addition, a temperature sensing element for sensing a temperature inside a body of a person wearing the catheter or cannula, which is operationally connectable to the output unit, wherein the output unit is structured and arranged for receiving from the temperature sensing element signals related to a temperature sensed by the temperature sensing element referred to as temperature signals, and for outputting signals referred to as temperature output signals, wherein the temperature output signals are temperature signals or signals derived from temperature signals.

85. The catheter or cannula arrangement according to claim 82, comprising an energy storage unit for supply of at least one of the sensing unit and the output unit with electrical energy, and wherein the output unit comprises a data storage unit, wherein at least one of
in the data storage unit, a sequence of data is stored, wherein each of the data of the sequence is representative of signals related to the sensed flow of the fluid at a different time;
the output unit comprises a control unit structured and arranged for creating in the storage unit a sequence of data representative of a time development of signals related to the sensed flow of the fluid;
wherein the sensing element is a thermal flow sensor.

86. The catheter or cannula arrangement according to claim 73, wherein the sensing unit is devoid of an energy storage unit for supply of the sensing unit with electrical energy.

87. The catheter or cannula arrangement according to claim 73, wherein the sensing unit comprises a valve for blocking a flow of the fluid through the sensing unit.

88. A device for use with a catheter or cannula, the device comprising an output unit comprising
a first interface for receiving, in a wireless or in a wirebound fashion, signals related to a sensed flow of a fluid through the catheter or cannula referred to as sensed signals;
a second interface, for transmitting the sensed signals or signals derived from the sensed signals in a wireless or in a wirebound fashion;
an energy storage unit for supply of at least the output unit with electrical energy;
a data storage unit, wherein at least one of
in the storage unit, a sequence of data is stored, wherein each of the data of the sequence is representative of sensed signals sensed at a different time;
the output unit comprises a control unit structured and arranged for creating in the storage unit a sequence of data representative of a time development of sensed signals and for creating the sequence of data by subsequently storing in the storage unit data representative of sensed signals sensed at subsequent times.

89. The device according to claim 88, wherein the energy storage unit is present inside a housing of the output unit.

90. The device according to claim 88, wherein the output unit comprises one or more fixing structures for attaching the output unit to tubing of the catheter or cannula.

91. The device according to claim 88, wherein the output unit is devoid of at least one of
any visual display capable of graphically or alphanumerically displaying information;
any visual display structured and arranged for graphically or alphanumerically displaying a representation of sensed signals or of data derived from sensed signals.

92. An arrangement for use in sensing a flow of a fluid flowing through a catheter or cannula, the system comprising
a sensing unit comprising a sensing element for sensing a flow of a fluid; and
an output unit operationally connectable to the sensing unit, for receiving from the sensing unit signals related to the sensed flow of the fluid referred to as sensed signals, and for outputting signals referred to as outputted signals, wherein the outputted signals are sensed signals or are signals referred to as processed signals which are obtained by the output unit by processing the sensed signals;
wherein at least one of
the output unit comprises one or more fixing structures for attaching the output unit to another part of the catheter or cannula arrangement;
the output unit comprises an energy storage unit for supply of at least one of the sensing unit and the output unit with electrical energy, wherein the energy storage unit is present in a housing of the output unit;
the output unit comprises a data storage unit, wherein at least one of
in the storage unit, a sequence of data is stored, wherein each of the data of the sequence is representative of sensed signals sensed at a different time;
the output unit comprises a control unit structured and arranged for creating in the storage unit a sequence of data representative of a time development of sensed signals.

93. The arrangement according to claim 92, wherein the fixing structures are structured and arranged for attaching the output unit to tubing of the catheter or cannula

94. The arrangement according to claim 92, wherein the control unit is structured and arranged for creating the sequence of data by subsequently storing in the storage unit data representative of sensed signals sensed at subsequent times.
